(19) United States
(12) Patent Application Publication
(10) Pub. No.: US 2011/0178484 A1
(43) Pub. Date: Jul. 21, 2011

(54) Apparatus for collection of urine

(76) Inventor: Mark Zivley, Houston, TX (US)

(21) Appl. No.: 12/657,329

(22) Filed: Jan. 19, 2010

(51) Int. Cl. A61F 5/453

(52) U.S. Cl. 604/352

(57) ABSTRACT

An apparatus for collection of urine from a male wearer comprises a housing having an outlet and having an opening for insertion of the distal end of a wearer's penis therein. An elastic membrane within the housing is inflatable to establish a substantially annular seal around the wearer's penis, such that urine from the wearer is directed out the outlet of the collection apparatus. In one embodiment, the apparatus is comprised of off-the shelf PVC components with the elastic membrane being formed from a portion of a conventional condom or the like.

### FIELD OF THE INVENTION

The present invention relates generally to the collection of urine, and more particularly to the collection of urine from a human male.

### BACKGROUND OF THE INVENTION

There are certain activities and situations in which it is desirable to permit a man to urinate in such a manner that the urine is collected for later disposal. Oftentimes, such situations arise in which a man is occupied with tasks which necessarily prevent him from addressing a need to urinate in a conventional manner, e.g., using restroom facilities. One example of this arises in the context of piloting a glider or other airborne craft, where one's attention is effectively dominated by the operation and control of the craft. Another example is that of a long-haul trucker, whose attention must be fixed on operating a large truck for long, uninterrupted periods of time. Numerous other examples are believed to exist.

To address the need for collection and/or disposal of a person's urine, the prior art has shown one approach involving the use of a so-called “catheter” device attachable to a man's penis such that urine is collected and directed to a means of containment or disposal, typically through a flexible hose or the like. Examples of this are shown in U.S. Pat. No. 4,239,044 to Pavlinch, entitled “Inflatable Catheter for a Male Urinal;” U.S. Pat. No. 4,202,335 to Gold, entitled “External Catheter Drainage Device;” and U.S. Pat. No. 6,805,662 to Shah et al., entitled “Urinary Incontinence Control Device and Method of Use.” The '044 patent to Pavlinch (“Pavlinch”) is hereby incorporated by reference herein in its entirety.

In general, these prior art devices comprise a cuff or other such means for being engaged with some distal portion of a man's penis. Preferably, means are provided for sealingly engaging the distal portion of a penis such that during urination, urine is not allowed to escape, but instead is collected in some manner and conveyed to disposal and/or containment at another location.

Obviously, the penis is a sensitive portion of a man's anatomy, and one not inherently suited to engagement with artificial urine collection apparatus, particularly when a fluid-tight seal is to be maintained over the distal portion of a penis. Moreover, for the same reasons why a urine collection apparatus would be required in the first place, it is preferable that any such apparatus be simple to operate and be comfortable to wear, even over prolonged periods of use. At the same time, it is desirable for the apparatus be easily deployed, in situations where its use only when needed is preferred.

It is also desirable for a urine collection apparatus to be designed so as maintain a fluid-tight seal around a penis while at the same time avoiding problems with restriction of urine flow.

Also desirable is for a urine collection apparatus to be relatively inexpensive, lightweight, and, to the extent possible, hygienic in operation and storage.

### SUMMARY OF THE INVENTION

In view of the foregoing and other considerations, the present invention is directed to a urine collection apparatus for fluid-tight engagement with at least a distal portion of a man's penis.

In one embodiment of the invention, an apparatus is provided comprising a rigid outer body which carries an internal, flexible membrane. Means are provided to inject pneumatic pressure into the apparatus causing inwardly-directed inflation of the membrane around the perimeter of the penis. A distal outlet of the apparatus is provided for coupling to an external means for containment or disposal of urine by means of a flexible hose or the like.

In accordance with one aspect of the invention, the urine-collecting apparatus can be modular in construction enabling the internal membrane to be readily replaced in the event of damage or for hygienic reasons, for example.

In accordance with another aspect of the invention, the portion of the apparatus connectible to a penis is relatively compact and lightweight.

### BRIEF DESCRIPTION OF THE DRAWINGS

The present invention is best understood with reference to the following detailed description of embodiments of the invention when read in conjunction with the attached drawings, in which like numerals refer to like elements, and in which:

FIG. 1 is a side cross-sectional view of a urine collection apparatus in accordance with one embodiment of the invention;

FIG. 2 is a side cross-sectional view of a urine collection apparatus in accordance with an alternative embodiment of the invention.

### DETAILED DESCRIPTION OF A PREFERRED EMBODIMENT OF THE INVENTION

In the disclosure that follows, in the interest of clarity, not all features of actual implementations are described. It will of course be appreciated that in the development of any such actual implementation, as in any such project, numerous engineering and technical decisions must be made to achieve the developers' specific goals and subgoals (e.g., compliance with system and technical constraints), which will vary from one implementation to another. Moreover, attention will necessarily be paid to proper engineering practices for the environment in question. It will be appreciated that such development efforts might be complex and time-consuming, outside the knowledge base of typical laymen, but would nevertheless be a routine undertaking for those of ordinary skill in the relevant fields.

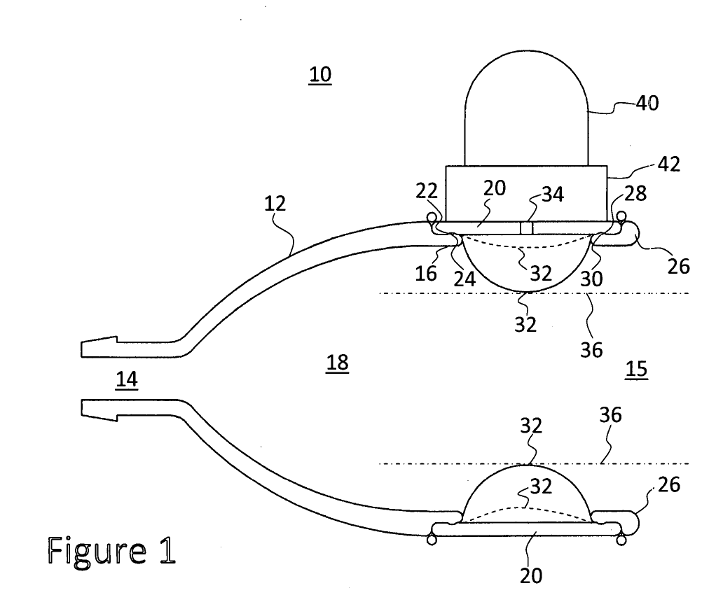

Referring to FIG. 1, there is shown a side cross-sectional view of a urine collection apparatus 10 in accordance with one embodiment of the invention. Apparatus 10 comprises main body 12 having a fluid outlet 14 of a relatively small diameter at a distal end thereof, and an open proximal end 15 of a relatively larger diameter.

In the presently disclosed embodiment, outer body 12 is rigid, preferably being made out of lightweight plastic such as (without limitation) acrylonitrile-butadiene-styrene (ABS) or polyvinyl chloride (PVC) plastic. Main body portion 12 defines an open internal space 18 of sufficient size to contain the distal end of a man's penis (not shown) therein.

Collection apparatus 10 further comprises a substantially annular spacer element 20 attachable to the proximal end of main body 12. In one embodiment, spacer 20 is configured to snap-fit with main body 12, with an internal detent 24 near the proximal end of body 12 engaging a corresponding external groove 22 on spacer 20 to secure body 12 and spacer 20 together.

A substantially annular rear retainer ring 26 similarly snap-fits onto the proximal end of spacer 20, with an internal detent 30 engaging a corresponding external groove 28 on spacer 20 to secure retainer 26 to spacer 20 together. Preferably, the snap-fit connections between spacer 20 and body 12, and between spacer 20 and retainer 26, are releasable, as will be hereinafter described.

In the presently disclosed embodiment, a flexible membrane 32 is folded around spacer 20 and is pinched within the snap-fit connection between spacer 20 and body 12 and within the snap-fit connection between spacer 20 and retainer 26, such that a portion of the membrane 32 is maintained against the inner surface of spacer 20. In one embodiment, membrane 32 is made of latex rubber. Membrane 32 is shown in inflated (solid line) and uninflated (dashed line) state in FIG. 1.

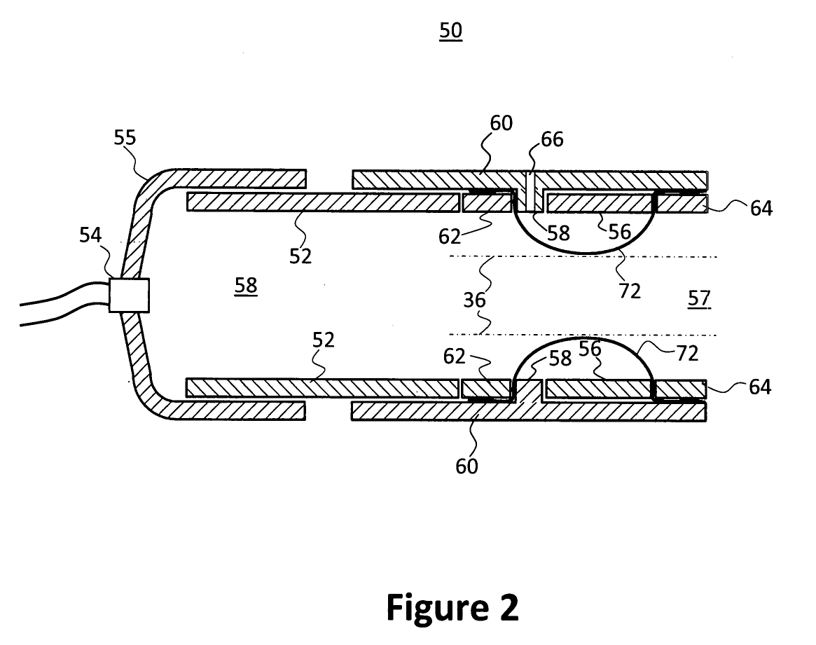

Preferably, at least one inflation hole 34 is provided through spacer 20. With this arrangement, introduction of inflation pressure into inflation hole 34 causes membrane 32 to inflate inwardly, forming a substantially annular sealing element as is shown in the side view of FIG. 2. By controlling and maintaining the internal pressure applied through inflation hole 34, a desired extent of inward inflation of membrane 32 can be achieved, such that the inflated membrane 32 can form a seal around the circumference 36 of a wearer's penis. By sealing in this manner, any urine expelled by the wearer will exit cavity 18 via outlet 14; urine is prevented from exiting from the proximal end of apparatus 10 due to the annular seal formed by inflated membrane 32 around the penis.

In fact, in the embodiment of FIG. 1, the apparatus 10 may be constructed from off-the-shelf ABS or PVS parts, including couplings, end caps, lengths of pipe, and the like, as would be apparent to those of ordinary skill in the art having the benefit of this disclosure.

Indeed, an embodiment of the invention comprising off-the-shelf parts is depicted in FIG. 2. Referring to FIG. 2, a urine collection apparatus 50 in accordance with one embodiment of the invention is shown in cross section. Apparatus 50 comprises main body 52 having a fluid outlet 54 in the form of a fitting extending through an end-cap 55 of a relatively small diameter at a distal end thereof, and an open proximal end 57 of a relatively larger diameter. In the embodiment of FIG. 2, main body 52 is formed of a length of PVC tubing of, for example, 1½-inch diameter, and cap 54 is a standard part adapted to terminate one end of a piece of PVC pipe.

In the embodiment of FIG. 2, outer body 52 is rigid, preferably being made out of lightweight plastic such as (without limitation) acrylonitrile-butadiene-styrene (ABS) or polyvinyl chloride (PVC) plastic. Main body portion 52 defines an open internal space 58 of sufficient size to contain the distal end of a man's penis therein.

Collection apparatus 50 further comprises a substantially annular spacer assembly comprising in part a coupling 60 attachable to the proximal end of main body 52. In one embodiment, as can be seen in FIG. 2, coupling 60 is configured to friction-fit with main body 52. coupling element 60 is, in the present embodiment, a conventional fitting for joining together two lengths of PVC pipe, as would be familiar to those of ordinary skill in the art.

A spacer ring 56 is fitted against the proximal side of a central flange 58 of coupling 60 (central flange 58 being a typical feature of an off-the-shelf PVC coupling). Spacer ring 56 is yet another relatively short length of PVC pipe.

A first substantially annular rear retainer ring 62, comprising a short length of PVC pipe, is fitted against the distal side of flange 58, and serves to secure a distal end of an annular membrane 72 against the inner wall of coupling 60. Likewise, a second retainer ring 64 is inserted into the proximal end of coupling 60 and secures a proximal end of membrane 72 against the inner wall of coupling 60.

Preferably, the friction fitting of retainer rings 62 and 64 within coupling 60 is such that the rings 62 and 64 can be removed when it is necessary to replace membrane 72. Spacer 56 can be permanently affixed in place, as it is not necessary to remove spacer 56 to replace membrane 72.

Preferably, at least one inflation hole 66 is provided through coupling 60, as shown in FIG. 2. With this arrangement, introduction of inflation pressure into inflation hole 66 causes membrane 72 to inflate inwardly, forming a substantially annular sealing element as is shown in the side view of FIG. 2. By controlling and maintaining the internal pressure applied through inflation hole 66, a desired extent of inward inflation of membrane 32 can be achieved, such that the inflated membrane 72 can form a seal around the circumference of a wearer's penis 36. By sealing in this manner, any urine expelled by the wearer will exit cavity 58 via outlet 54; urine is prevented from exiting from the proximal end of apparatus 50 due to the annular seal formed by inflated membrane 72 around the penis 36.

From the foregoing disclosure, it should be apparent that an apparatus for collecting urine from a human male has been disclosed. Although specific embodiments of the invention as well as possible variants and alternatives thereof have been described and/or suggested herein, it is to be understood that the present disclosure is intended to teach, suggest, and illustrate various features and aspects of the invention, but is not intended to be limiting with respect to the scope of the invention, as defined exclusively in and by the claims, which follow.

Indeed, it is contemplated and to be explicitly understood that various substitutions, alterations, and/or modifications, including but not limited to any such implementation variants and options as may have been specifically noted or suggested herein, including inclusion of technological enhancements to any particular method step or system component discovered or developed subsequent to the date of this disclosure, may be made to the disclosed embodiment of the invention without necessarily departing from the technical and legal scope of the invention as defined in the following claims.

What is claimed is:

1. An apparatus for collecting urine from a male wearer, comprising:
a main body having a distal drainage port;
a substantially annular spacer element adapted to connect with a proximal end of said main body, and to circumferentially secure one edge of an annular membrane;
a substantially annular retainer element adapted to connect with a proximal end of said spacer element and to circumferentially secure another edge of said annular membrane, such that said membrane is exposed on the interior face of said spacer element;
at least one inflation hole through said spacer element, for directing inflation pressure between said spacer element and said membrane, thereby inflating said membrane radially inward from said interior face of said spacer element;
said membrane, when inflated, forming an annular fluid-tight seal around a portion of the penis of a wearer, such that urine expelled by said wearer is directed forward and out through said drainage port.
